(19) United States
(12) Patent Application Publication
(10) Pub. No.: US 2010/0178346 A1
(43) Pub. Date: Jul. 15, 2010

(54) DISC AUGMENTATION WITH HYALURONIC ACID

(75) Inventors: Neil R. Malhotra, Philadelphia, PA (US); Weiliam Chen, Mount Sinai, NY (US); Dawn M. Elliott, Philadelphia, PA (US)

Correspondence Address: WOODCOCK WASHBURN LLP, CIRA CENTRE, 12TH FLOOR, 2929 ARCH STREET, PHILADELPHIA, PA, 19104-2891, US

(73) Assignee: THE TRUSTEES OF THE UNIVERSITY OF PENNSYLVANIA, Philadelphia, PA (US)

(21) Appl. No.: 12/524,782

(22) PCT Filed: Feb. 1, 2008
(86) PCT No.: PCT/US2008/001403

§ 371 Date: Mar. 5, 2010

Related U.S. Application Data
(60) Provisional application No. 60/899,139, filed on Feb. 1, 2007.

(51) Int. Cl. A61K 9/00; C08B 37/08; A61K 31/728; A61K 35/12; A61K 38/02; A61K 49/00; A61P 19/00

(52) U.S. Cl. 424/488; 536/55.1; 514/54; 424/93.7; 514/2; 424/9.1

(57) ABSTRACT

The present invention relates to materials comprising hyaluronic acid for replacement or augmentation of the nucleus pulposus The materials have a Poisson's ratio of 0.40 to 0.80 based on that of the human nucleus pulposus.

### CROSS-REFERENCE TO RELATED APPLICATIONS

This Application claims the benefit of U.S. Provisional Application No. 60/899,139, filed Feb. 1, 2007, the entirety of which is incorporated herein by reference.

### FIELD OF THE INVENTION

The present invention relates to materials useful for replacement or augmentation of the nucleus pulposus, as well as methods of using those materials. In particular, hyaluronic acid-based materials are described.

### BACKGROUND OF THE INVENTION

Degenerative disc disease (“DDD”) is an irreversible condition and is the leading cause of pain and disability in American adults. Traditional treatment for discogenic pain involves discectomy and spinal fusion, which may relieve pain but result in the loss of disc mechanical function and may lead to DDD in adjacent segments. Moreover, microdiscectomy results in the loss of nucleus pulposus tissue that can alter mechanical function of the intervertebral joint, leading to progressive disc degeneration and potentially stressing adjacent vertebra. Meakin J R, Hukins D W: Effect of removing the nucleus pulposus on the deformation of the annulus fibrosus during compression of the intervertebral disc. J Biomech 33:575-580, 2000; Seroussi R E, Krag M H, Muller D L, et al: Internal deformations of intact and denucleated human lumbar discs subjected to compression, flexion, and extension loads. J Orthop Res 7:122-131, 1989.

Nucleus pulposus (“NP”) replacement is a non-fusion technique currently being investigated to treat DDD. Wilke H J, Kavanagh S, Neller S, et al: Effect of a prosthetic disc nucleus on the mobility and disc height of the L4-5 intervertebral disc postnucleotomy. J Neurosurg 95:208-214, 2001. Replacement of NP with a nuclear prosthetic or a tissue-engineered construct in patients with healthy annulus fibrosus may reduce pain while simultaneously restoring spinal mobility and delaying disc degeneration.

One goal of developing a nucleus pulposus replacement is to improve range of motion; an increase in range of motion is an indication of diminished nucleus pulposus. Degenerative changes of the annulus fibrosus are likely subsequent to, and a result of, increased strain following altered load transfer from the NP. In degeneration or microdiscectomy, load transfer changes from NP occur via a loss of glycosaminoglycan content, as well as loss of nucleus pulposus pressure, which results in increased range of motion. Worsening function of the disc is manifested by the outer annulus bulging outward, and inner annulus inward, when under axial load. The inner and outer annulus bulging provokes circumferential annular tears that further progress joint insufficiency. Reversal of increased range of motion, i.e., a decrease in range of motion, is therefore an indicator of revitalized NP function. Wilke H J, Kavanagh S, Neller S, et al: Effect of a prosthetic disc nucleus on the mobility and disc height of the L4-5 intervertebral disc postnucleotomy. J Neurosurg 95:208-214, 2001.

A complete description of human nucleus pulposus (NP) mechanics is critical for evaluating potential nucleus pulposus replacement materials. These materials should function mechanically similar to and mimic the properties of native NP tissue. But the challenge for any synthetic nucleus replacement material is how to identify which properties of native NP to mimic. The functional mechanics of NP tissue are varied and include such properties as an incompressible fluid, a poroelastic material, an isotropic solid, and a biphasic material. Moreover, the NP behaves physiologically in an environment that is neither completely confined nor completely unconfined: in vivo, the NP is confined axially by the superior and inferior cartilaginous end plates and circumferentially by the annulus fibrosus, with compressive loads on the NP transferred to the annulus when the NP distends radially. In degeneration, the NP fails to transfer loads between the vertebral bodies due to failure to maintain annular fibers in tension.

While certain properties of human NP in confined compression have been reported (Johannessen et al., Spine, 2005. 30 (24):E724), the unconfined compression properties, namely equilibrium modulus and Poisson's ratio, are not known and are key functional parameters for NP replacement. Previous studies have relied on theoretical values of Poisson's ratio for NP; Poisson's ratio has not previously been measured directly for human NP.

These varied and unknown properties of native NP have impeded efforts to develop a synthetic NP replacement that can successfully mimic native NP. Thus, what are needed are materials that mimic native NP properties. What are also needed are materials that reverse increased range of motion following discectomy and disc degeneration.

### SUMMARY OF THE INVENTION

Sterile, biocompatible materials for the replacement or augmentation of the nucleus pulposus in an intervertebral disc, comprising, in major proportion, hyaluronic acid, hyaluronic acid derivative, or mixtures thereof, having a Poisson's ratio of between about 0.40 to about 0.80, are described herein.

### BRIEF DESCRIPTION OF THE DRAWINGS

FIG. 1 depicts one embodiment of an unconfined compression testing configuration useful for testing materials of the present invention. The configuration consists of an optically translucent testing surface with space for mounting of a digital camera.

FIG. 2 (A) Schematic of stress-relaxation experiment performed according to one embodiment of the present invention, consisting of incremental steps of 5% strain followed by a five minute relaxation period to a total of 25% strain. (B) Average equilibrium stress-strain curves produced using mean A and B values curve fit according to the equation σ=A(e^{βε}−1).C) Representative linear regression for calculation of Poisson's ratio.

FIG. 3 Comparison of material properties in unconfined compression using procedures according to one embodiment of the present invention. NP, nucleus pulposus; A, B, and C, three experimental hyaluronic acid-based hydrogels; alg, 2.0% medium viscosity alginate; aga, 2.0% med viscosity agarose. A) toe modulus calculated at 0% strain B) Linear region modulus calculated at 20% strain C) Poisson's ratio D) relaxation (%)*significantly different than human NP (P<0.05).

FIG. 4 Cross sectional SEMs of materials prepared according to the present invention. (A) oHA-gelatin (4:6). (B) oHA-gelatin (5:5). (C) oHA-gelatin 6:4. (D) pure gelatin.

FIG. 5 Unconfined compression equilibrium properties and relaxation for NP and materials prepared according to the present invention. *=significantly different than human NP p<0.05

FIG. 6A Comparison of compressive stiffness following microdiscectomy compared to control.

FIG. 6B Comparison of tensile stiffness following microdiscectomy compared to control.

FIG. 6C Comparison of range of motion following microdiscectomy compared to control.

FIG. 7 Comparison of range of motion of control, after microdiscectomy, and after injection of an exemplary material of the present invention (Hydrogel C).

### DETAILED DESCRIPTION OF ILLUSTRATIVE EMBODIMENTS

The present invention is directed to sterile, biocompatible materials for the replacement or augmentation of the nucleus pulposus in an intervertebral disc. Such materials have a Poisson's ratio that approximates the Poisson's ratio of human nucleus pulposus, the determination of which is described herein. It has now been found beneficial to prepare materials of the present invention having controlled Poisson's ratios. In particular, Poisson's ratios of between about 0.4 and about 0.8 for these materials have been found to be particularly beneficial. Preferably, the materials have a Poisson's ratio of between about 0.45 and 0.70. In other preferred embodiments, the materials have a Poisson's ratio of between about 0.43 and 0.69. Most preferably, the materials have a Poisson's ratio of between about 0.55 and about 0.65. Particularly preferred are those materials having a Poisson's ratio of about 0.62 or about 0.56.

In preferred embodiments, the materials of the present invention comprise, in major proportion, hyaluronic acid, hyaluronic acid derivative, or a mixture thereof. Hyaluronic acid is also referred to by those of skill in the art as hyaluronan or hyalurononate. Materials of the present invention may further comprise water or other physiologically compatible liquids. It is envisioned that in certain embodiments of the present invention, water, or other physiologically compatible liquid, may be present in amounts up to about 90% by volume of the total composition.

In materials of the present invention, the major proportion of hyaluronic acid, hyaluronic acid derivative, or mixtures thereof is from about 5% to about 20% by weight of the material. Preferably, the major proportion of hyaluronic acid, hyaluronic acid derivative, or mixtures thereof is at least about 10% by weight of the material. Particularly preferred are those embodiments wherein the major proportion of hyaluronic acid, hyaluronic acid derivative, or mixtures thereof is about 14% by weight of the material. In some embodiments, the major proportion of hyaluronic acid, hyaluronic acid derivative, or mixtures thereof, is at least about 30% by weight of the material. In other the major proportion is at least about 40% by weight of the material. In yet other embodiments, the major proportion of hyaluronic acid, hyaluronic acid derivative, or mixtures thereof, is at least about 50% by weight of the material. In certain embodiments, the major proportion is at least about 60% by weight of the material. In still other embodiments, the major proportion is at least about 70% by weight of the material.

Preferably, the materials of the present invention are hydrogels. In certain preferred embodiments, the materials further comprise a biocompatible polymer, for example, gelatin, collagen, polysaccharides, or mixtures thereof. In those embodiments further comprising gelatin, the major proportion of hyaluronic acid, hyaluronic acid derivative, or mixtures thereof, added to the proportion of gelatin is at least about 30% by weight of the material. In those embodiments further comprising collagen, the major proportion of hyaluronic acid, hyaluronic acid derivative, or mixtures thereof, added to the proportion of collagen is at least about 30% by weight of the material. In those embodiments further comprising gelatin and collagen, the major proportion of hyaluronic acid, hyaluronic acid derivative, or mixtures thereof, added to the proportion of collagen is at least about 30% by weight of the material.

In those embodiments further comprising gelatin, in addition to hyaluronic acid, hyaluronic acid derivative, or mixtures thereof, the ratio of hyaluronic acid, hyaluronic acid derivative, or mixtures thereof to gelatin is from about 2:8 to about 8:2. Particularly preferred are those wherein the ratio is from about 3:7 to about 7:3. Most preferably, the ratio is 7:3.

In other embodiments, the materials may further comprise medicaments, for example analgesics and neuropeptide receptor competitive inhibitors. In yet others, the materials may further comprise cells, growth hormones, antibiotics, cell signaling materials, or a plurality thereof. In still others, the materials may further comprise imaging contrast agents, such as for example, gadolinium-containing compounds.

In exemplary embodiments of the present invention, the materials comprise hyaluronic acid derivatives. Hyaluronic acid derivatives include, for example, oxidized hyaluronic acid and partially oxidized hyaluronic acid. The physical properties of the materials of the present invention may be optionally modified by the addition of further substances, for example, carboxymethylcellulose.

The materials of the present invention may also be porous. In certain embodiments, the pore sizes of such porous materials is between about 20 μm and about 90 μm.

One exemplary embodiment of the present invention comprises a porous, intervertebral disc augmentation or replacement material comprising at least 30% by weight of the material hyaluronic acid, hyaluronic acid derivative, or a mixture thereof, in optional admixture with gelatin, collagen or both, the porous material having a Poisson's ratio of between about 0.40 to about 0.80. Preferably, the material comprises about 50% by weight the material hyaluronic acid, hyaluronic acid derivative, or a mixture thereof, in optional admixture with gelatin, collagen or both, the porous material having a Poisson's ratio of between about 0.40 to about 0.80. In preferred embodiments, the material comprises a Poisson's ratio of from about 0.55 to about 0.65, with a ratio of about 0.62 or 0.56 being most preferred.

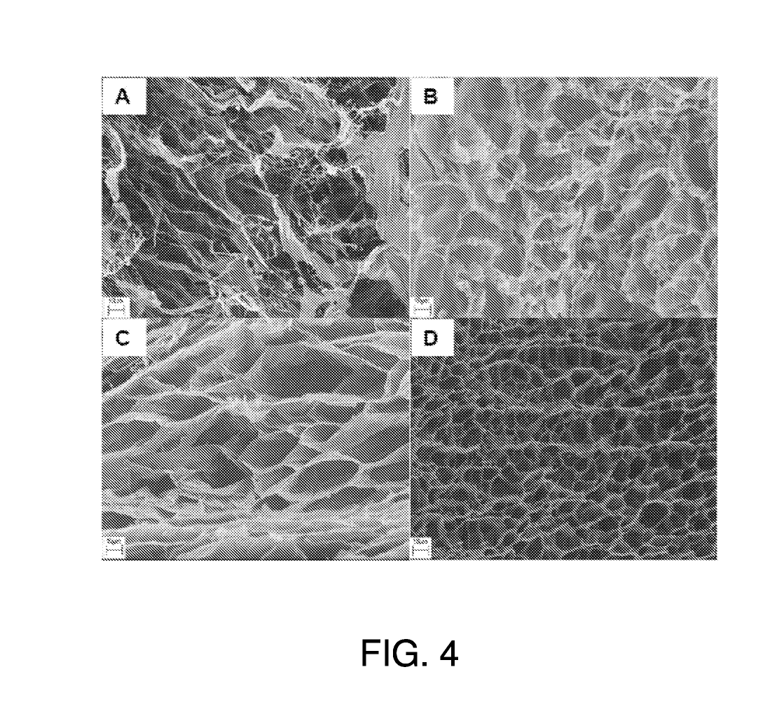

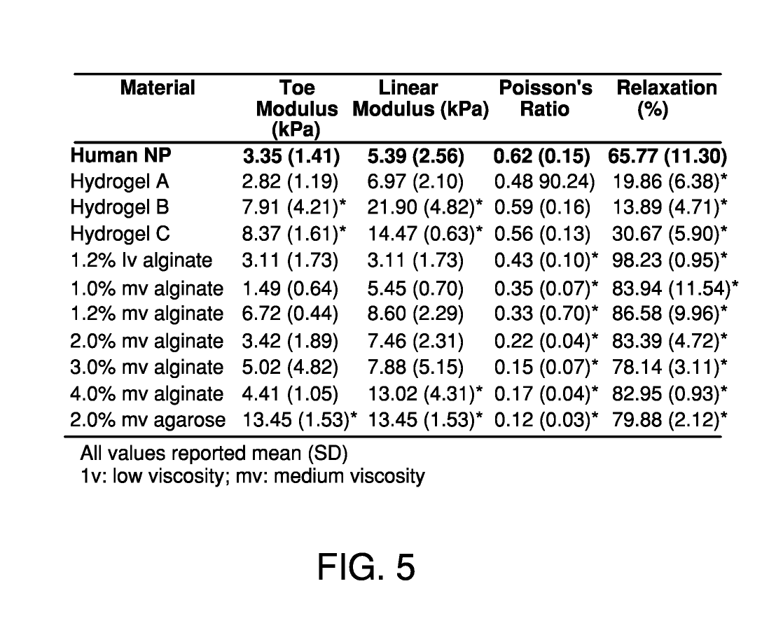

Properties of human NP, for example, compression modulus and Poisson's ratio, in unconfined compression, are described herein. The healthy nucleus pulposus operates through the Poisson effect of an elastomeric synthetic material under load. Poisson's ratio (υ) is the ratio of lateral strain divided by axial strain (υ=εₗ/εₐ). Using the methods described herein, physical properties of human NP, including Poisson's ratio, have been determined. (FIG. 5). Poisson's ratio for certain hyaluronic acid-gelatin based materials has also been determined. (FIG. 5)

While the present invention has been particularly shown and described with reference to the presently preferred embodiments thereof, it is understood that the invention is not limited to the embodiments specifically disclosed herein. Numerous changes and modifications may be made to the preferred embodiments of the invention, and such changes and modifications may be made without departing from the spirit of the invention. It is therefore intended that the appended claims cover all such equivalent variations as they fall within the true spirit and scope of the invention.

The instant invention is illustrated by the following examples that are not intended to limit the scope of the invention.

### EXAMPLES

### Materials

Hyaluronan (Mw 1.5×10⁶) was from Engelhard, Inc. (Stony Brook, N.Y.). Gelatin (Bloom 300, Type A, Mw 100,000), sodium periodate, tetraborate decahydrate (borax) were purchased from Signma-Aldrich (St. Louis, Mo.). Dialysis tube (MWCO 3,500) was from Fisher (Hampton, N.H.). All other chemicals were of reagent grade. Deionized and distilled water was used.

### Example 1

### Determination of Poisson's Ratio for Human NP

A. Preparation of Human NP Sample

Five human lumbar spines (3 male, 2 female; age range 19-76 years, median 25 years) were obtained from IRB approved tissue sources (National Disease Research Interchange, Philadelphia, Pa. and International Institute for the Advancement of Medicine, Jessup, Pa.). Healthy lumbar intervertebral discs were removed from each spine via sharp dissection along the superior and inferior end plates. A 12.7 mm punch was removed from the nuclear region and microtomed to a uniform thickness of 5 mm. A 7.1 mm sample was subsequently punched from the removed region, wrapped in plastic wrap and stored at −20° C. until the time of mechanical testing.

B. Testing of NP Samples

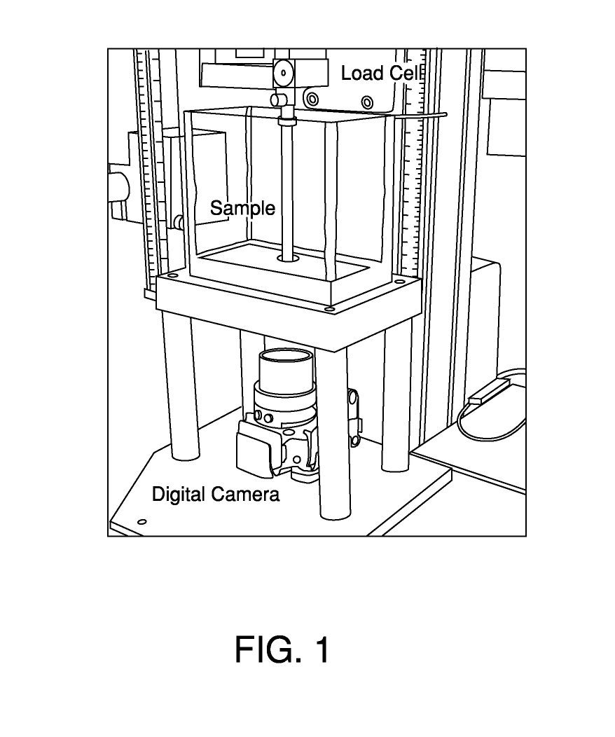

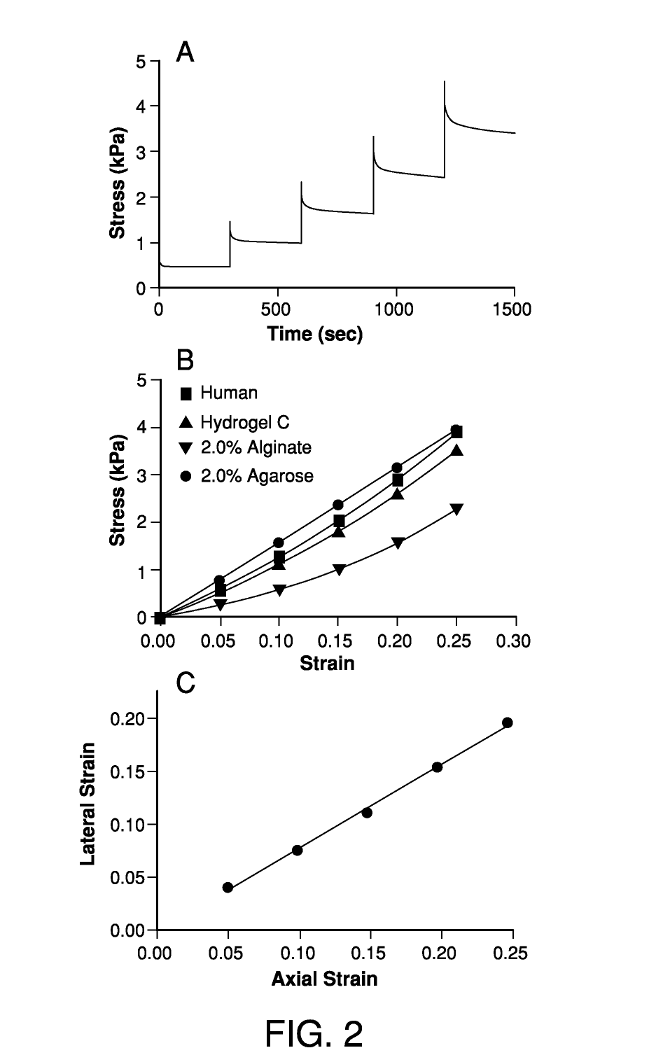

A tank made entirely of clear acrylate on a 15 cm tall platform was constructed and a digital camera (Canon Powershot S21S) was mounted directly underneath the testing surface (FIG. 1). The previously prepared NP samples were tested in a phosphate buffered solution. A flat, non-porous plunger was attached to an Instron mechanical testing system (Instron 5542, Canton, Mass.) fitted with a 5N load cell. The plunger was lowered to make contact with the platform base to zero the instrument displacement. NP samples, stained with blue dye to permit improved optics, were then placed on the platform and a 0.05N preload was applied for 10 minutes. The load was removed and the sample was allowed to equilibrate for 5 minutes to allow swelling. An incremental stress-relaxation test was then performed, consisting of 5% strain increments at a rate of 5%/sec, followed by 5 minutes of relaxation. Strain increments were repeated to 25% strain (FIG. 2A). Load and displacement data were recorded at 100 Hz. At the end of each relaxation period, a digital image of the sample was acquired from below for lateral strain analysis.

C. Calculation of Poisson's Ratio

The non-linear stress-strain data at the end of each relaxation period were curve fit according to the equation σ=A(e^{βε}−1) (Graphpad Prism 4.0). Toe region modulus was calculated as the slope of this curve at 0% strain. Linear region modulus was calculated as the slope of the stress-strain curve at 20% strain. Percent relaxation was calculated as σₑ/σₚ×100%, where σₑ is the equilibrated stress and σₚ is the peak stress. Strain analysis was performed using image software (Uthscsa Image Tool 3.0) to calculate lateral strain (εₗ) as the change in diameter over the initial diameter of the cylindrical sample. Axial strain (εₐ) was calculated by the change in crosshead displacement over initial height. Poisson's ratio was calculated using a linear regression of the εₗ-εₐ data (5 points). The set-up was confirmed by testing commercial rubber (υ=0.51±0.16) which has a Poisson's ratio of 0.5.

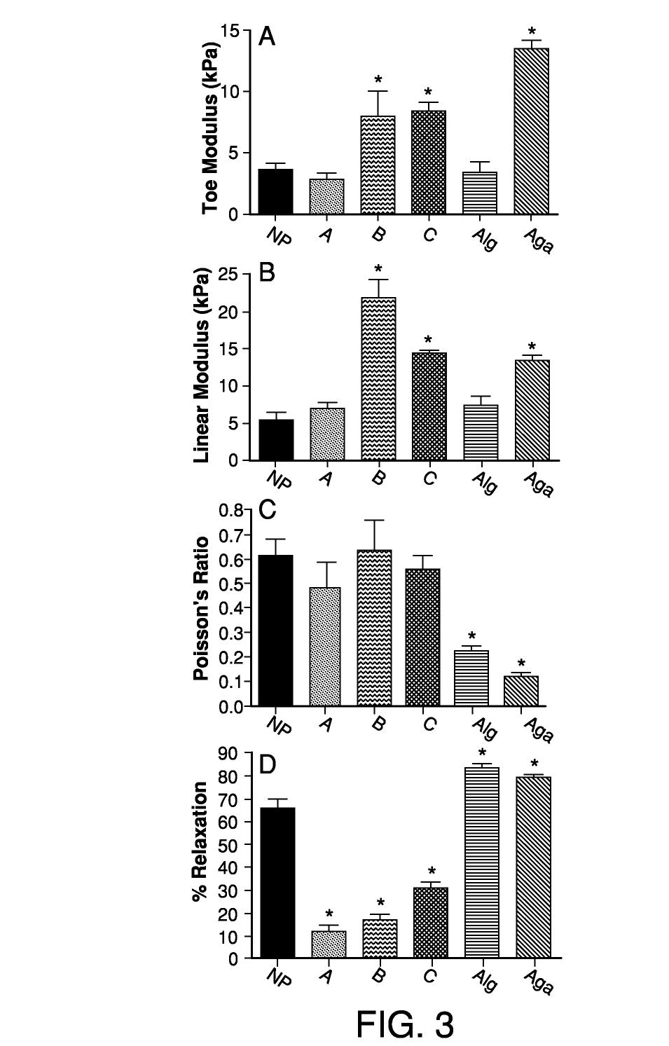

Curve fit of the stress-relax data (FIG. 3) for unconfined compression experiments of human NP produced a good fit (FIG. 2B, r²>0.99, n=5). Toe modulus at equilibrium was 3.35±1.41 kPa, while linear region modulus was 5.39±2.56 kPa. Linear regression of the εₗ-εₐ data to calculate Poisson's ratio was well fit (FIG. 2C, r²>0.95, n=55). Poisson's ratio for human NP was thus calculated to be 0.62±0.15 and percent relaxation was 65.8±11.3%.

### Example 2

### Preparation of Hydrogel A

Hydrogel A was prepared by first blending 0.9 mL of 1% hyaluronic acid solution with an equal volume of 7% PEG-g-chitosan solution (extent of PEG grafting: 48%); gelation was initiated by swiftly mixing in 70 μL of a 17% ethyl-3-[3-dimethyl amino] propyl carbodiimide solution with an equal volume of a 6.5% N-hydroxysuccinimide solution. Samples of hydrogel A were prepared for mechanical testing by using a 7.1 mm cylindrical punch.

### Example 3

### Preparation of Hydrogel B

Hydrogel B was prepared according to the method of Example 1, except the concentration of hyaluronic acid solution was 2.6%. Samples of hydrogel B were prepared for mechanical testing by using a 7.1 mm cylindrical punch.

### Example 4

### Preparation of Hydrogel C

A. Preparation of Partially Oxidized Hyaluronan

One gram of hyaluronan was dissolved in 80 mL of water in a shaded flask and sodium periodate solutions (various amounts dissolved in 20 mL water, pH=5.4) were added dropwise to the hyaluronan solution in order to produce oxidized hyaluronan (oHA) with different degrees of oxidation. The reaction mixture was incubated and stirred at ambient temperature for a period of time. At the conclusion of the reaction, 10 mL of ethylene glycol was added to quench the reaction, followed by continual stirring at room temperature for an hour. The mixture was dialyzed exhaustively for 3 days against DD water, and pure oHA was obtained by lyophilization (typical yield: 50-67%). The degree of oxidation can be determined by ¹H NMR. The molecular weights of oHA (dissolved in water, concentration: 0.5 mg/mL) could be determined by HPLC (Waters Ultrohydrogel 2000, 1000, and 500; 300 mm×7.8 mm columns connected in series, 0.1 M KNO₃ as a mobile phase, flow rate of 0.8 mL/min, temperature 50° C.).

B. Blending of oHA and Gelatin

oHA and gelatin solutions of 20% (w/v) concentration (both dissolved in 0.1 M borax) were prepared, separately, at 37° C. oHA/Gelatin hydrogels were formulated by rapidly mixing pre-determined volumes of both solutions and it was injected directly into a mold designed to generate 7.1 mm diameter hydrogel samples for mechanical testing.

### Example 5

### Preparation of oHA-Gelatin Hydrogels

In accordance with Example 3, oHA-gelatin hydrogels of varying weight ratios can be prepared. oHA (prepared according to Example 3) and gelatin were mixed according to Example 3 in weight ratios of 3:7, 4:6, 5:5, 6:4, and 7:3, stirred for 1 min at 37° C. and incubated at 37° C. for up to 12 h to form hydrogels.

The hydrogel resulting from the 7:3 mixture of oHA:gelatin has a toe-region modulus of 8.31 kPa, linear modulus of 14.47 kPa, a Poisson's ratio of 0.56 and a relaxation percentage of 30.67.

### Example 6

### Porosity of oHA-Gelatin Hydrogels

Typical cross-sectional SEM images of lyophilized hydrogel formulations prepared from oHA-gelatin hydrogels of 27.8% degree of oxidation are depicted in FIG. 4A-C. FIG. 4D depicts a cross-sectional SEM of gelatin. The highly porous structure of the oHA-gelatin hydrogels (dimension 20 to 90 μm, average 60 μm), provides that the hydrogels will accommodate cell migration/cell infiltration.

### Example 7

### Preparation of Alginate Gels

Either low or medium viscosity sodium salt alginic acid (Sigma Chemical Co., St. Louis, Mo.) was used in all alginate gels. Alginate solutions, in concentrations between 1% and 4% by weight, were solubilized in deionized water. Molds were placed in a six-well plate, filled with the alginate solution, and immersed in 100 mM CaCl₂ solution for 18 hours. Cylindrical punches 7.1 mm in diameter were removed from the alginate gels and microtomed to a uniform thickness.

### Example 8

### Preparation of Agarose Gels

2% medium-viscosity agarose (Sigma Chemical Co., St. Louis, Mo.) by weight was cast in molds and allowed to cool overnight. All gels were then wrapped in plastic wrap and stored at 4° C. until mechanical testing

### Example 9

### Preparation of Ovine Lumbar Motion Segments

### Preparation 1

Ten lumbar spine motion segments were harvested from 5 skeletally mature sheep and dissected using IACUC approved protocol. Five (5) L3-L4 and five (5) L4-L5 motion segments were cut at the midpoint of the superior and inferior vertebral body. Bone-disc-bone units were potted in bone cement. Kirchner wires (2 per segment) were drilled through vertebral bodies and bone cement to increase pull-out strength. Samples were hydrated for 18 hours in a refrigerated PBS bath prior to each phase of mechanical testing.

### Preparation 2

Lumbar spines were harvested from 10 skeletally mature sheep spines previously obtained for a non-spine animal study. All musculature and soft tissue were dissected and facets and transverse processes were removed. Bone-disc-bone motion segments were prepared by making parallel cuts through the vertebral bodies above and below the disc at lumbar spine levels L3-4 and L4-5. Fourteen Motion segments were randomly selected (n=14) and potted in polymethyl methacrylate bone cement. Kirschner wires were placed through the bone cement and vertebral body to increase pull out strength. Motion segments were wrapped in saline soaked gauze throughout processing to prevent dehydration. Samples were randomly selected from the L3-4 and L4-5 levels to provide 5 from each level (n=10) for the treatment group and four motion segments (n=4) for the nontreatment group.

See generally, Wilke H J, Kettler A, Claes L E: Are sheep spines a valid biomechanical model for human spines? Spine 22:2365-2374, 1997.

### Example 10

### Mechanical Testing Procedure

Mechanical testing was divided into three phases for treatment and nontreatment groups. The first phase, the intact control phase, consisted of a cyclic tension compression protocol performed on motion segments divided into two groups, treatment (n=10) and nontreatment (n=4). Phase 2, the microdiscectomy phase, consisted of the same mechanical testing protocol as phase 1 but all motion segments, treatment and nontreatment group, had undergone microdiscectomy. For Phase 3, injection versus noninjection, all motion segments were subjected to the same mechanical testing protocol as phase 1 and phase 2, however, the treatment group had been treated with the injectable hydrogel implant and the nontreatment group received no injection.

Mechanical tests were performed in PBS bath on an Instron 880 or Instron 8874 servohydrolic test frame (Instron, Canton, Mass.). Each sample underwent mechanical testing: intact pre-surgery (CTL), post-microdiscectomy (MD), and post hydrogel injection (INJ). Each phase of mechanical testing included an axial cyclic compression-tension protocol: 20 cycles at 1 Hz, peak loads −300 N (compression; representing 1.5 times human body weight scaled for differences in cross sectional area of the human and ovine intervertebral discs. Elliott D M, Sarver J J: Young investigator award winner: validation of the mouse and rat disc as mechanical models of the human lumbar disc. Spine 29:713-722, 2004; O'Connell G D, Vresilovic E J, Elliott D M: Comparison of animals used in disc research to human lumbar disc geometry. Spine 32:328-333, 2007.) to +300 N (tension). Subsequent to mechanical testing, each sample underwent a freeze thaw cycle prior to the next test.

Microdiscectomy consisted of annular incision followed by partial nucleotomy from a posterior approach using standard surgical instruments. Annular incisions with an 11-blade were approximately 2.5 mm in diameter. A blunt probe was placed into the nuclear cavity to confirm transannular incision. Microrongeur was inserted into the nuclear cavity and loose nucleus pulposus material was removed (0.0243±0.003 g of NP removed, approximately 35% of the total NP).

Hydrogel injection was performed via a blunt 18 g or 21 g needle through annulotomy site. Nuclear cavity's were filled to annular opening site with hydrogel (0.26±0.09 ml) by hand pressure to syringe plunger only. The annulus injury was not repaired or filled with implant material. Motion segments were incubated at ambient body temperature (37° C.) for one hour to permit gel formation and then frozen until testing.

### Data Analysis

The first 19 cycles were assumed to be preconditioning and the 20^{th} cycles were analyzed. Compressive stiffness and tensile stiffness were taken as the slope of the load displacement data from −200 to −300N compression and +200 to 300 N for tension. Range of motion (ROM) was computed as the total peak to peak displacement. Effect of microdiscectomy and treatment on motion segment mechanics were analyzed using paired t-test with significance set at p<0.05.

### Example 11

### Ovine Microdiscectomy

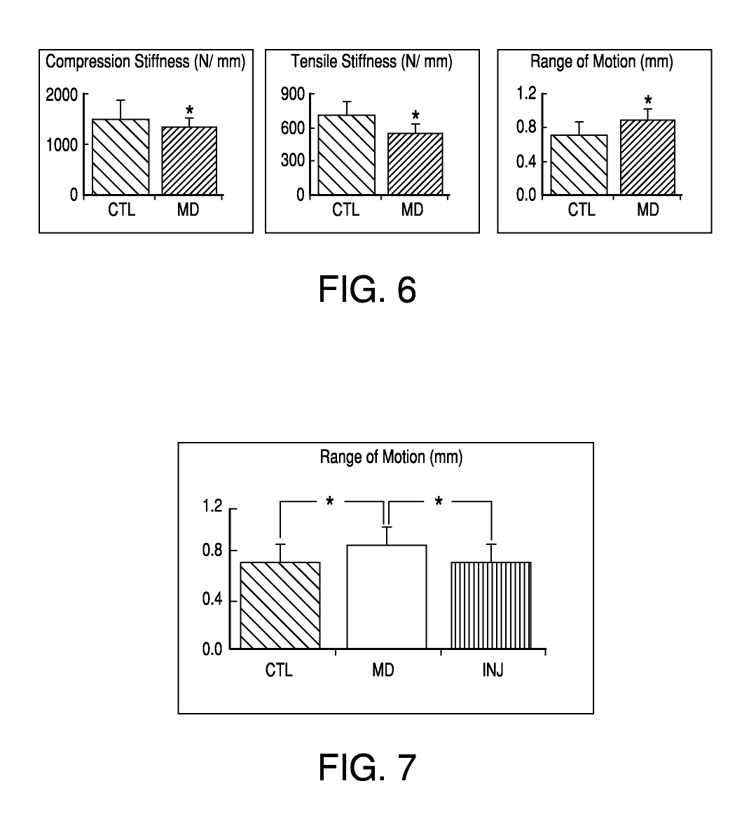

Microsurgical discectomy results in a 23% reduction in tensile stiffness (p<0.01) and an 11.4% reduction in compressive stiffness (p=0.088) when compared to intact control (FIGS. 6A-6B). Microdiscectomy led to an 18.4% (p<0.05) increase in range of motion (ROM) (FIG. 6C). This study demonstrates that microsurgical discectomy performed in vitro on the ovine motion segment results in a defined, statistically significant, decline in motion segment mechanics.

### Example 12

### Injection of Hydrogel C into Ovine Lumbar Motion Segments and ROM Determination

Hydrogel C was prepared according to Example 4 and was injected into the ovine NP cavity according to Example 9. No NP material or injectable hydrogel was observed to be ejected from the NP cavity during mechanical testing. ROM following NP injury (microdiscectomy) is different, i.e., increased, when compared to pre-surgical untreated control. ROM following treatment is reduced toward normal, native ROM when compared to ROM following microdiscectomy. There is no significant difference between control untreated motion segment ROM and ROM of those having undergone hydrogel C injection subsequent to NP injury. NP injections following microdiscectomy reduced ROM 17.2% (p<0.01) versus post surgical ROM. Further, injection returned ROM to pre-surgical levels (pre-surgery 0.71 mm, post injection 0.72 mm (p=0.08) (FIG. 7). Compressive stiffness decreased 9% (p<0.05) compared to MD. Tensile stiffness was unchanged.

What is claimed is:

1. A sterile, biocompatible material for the replacement or augmentation of the nucleus pulposus in an intervertebral disc comprising, in major proportion, hyaluronic acid, hyaluronic acid derivative, or a mixture thereof, the material having a Poisson's ratio of between about 0.40 to about 0.80.

2. The material of claim 1 in the form of a hydrogel.

3. The material of claim 1 further comprising gelatin, collagen, or a mixture thereof.

4. The material of claim 1 wherein the hyaluronic acid derivative is oxidized hyaluronic acid.

5. The material of claim 1 wherein the hyaluronic acid derivative is partially oxidized hyaluronic acid.

6. The material of claim 1 having a Poisson's ratio of between about 0.45 and 0.70.

7. The material of claim 1 having a Poisson's ratio of about 0.55 to about 0.65.

8. The material of claim 1 further comprising polymer.

9. The material of claim 1 further comprising medicament.

10. The material of claim 1 further comprising living cells, growth hormone, antibiotic, cell signaling molecules, or a plurality thereof.

11. The material of claim 1 further comprising an imaging contrast agent.

12. The material of claim 1 wherein the material is porous.

13. The material of claim 2 wherein the hydrogel is porous.

14. The material of claim 13 wherein the pore size is between about 20 and 90 micrometers.

15. The material of claim 1 wherein said major proportion is from about 5% to about 20% by weight of the material.

16. The material of claim 1 wherein said major proportion is about 14% by weight of the material.

17. The material of claim 3 wherein the material further comprises gelatin and wherein the ratio of hyaluronic acid, hyaluronic acid derivative, or a mixture thereof to gelatin is from about 2:8 to about 8:2.

18. The material of claim 3 wherein the material further comprises gelatin and wherein the ratio of hyaluronic acid, hyaluronic acid derivative, or a mixture thereof to gelatin is about 7:3.

19. A porous, intervertebral disc augmentation or replacement material comprising at least 5% by weight of the material of hyaluronic acid, hyaluronic acid derivative, or a mixture thereof, in optional admixture with gelatin, collagen or both, the porous material having a Poisson's ratio of between about 0.40 to about 0.80.

20. A sterile, biocompatible material for the replacement or augmentation of the nucleus pulposus in an intervertebral disc, the material having a Poisson's ratio of between about 0.40 to about 0.80.

21. A method of replacing or augmenting the nucleus pulposus in an intervertebral disc, comprising administering an effective amount of a biocompatible, sterile material, the material having a Poisson's ratio of between about 0.40 to about 0.80, to a patient in need thereof.

22. A kit for the replacement or augmentation of an interverbetral disc comprising a delivery device for injecting a thixotropic liquid, the liquid having a Poisson's ratio of between about 0.40 to about 0.80, into the space normally occupied by the nucleus pulposus in the spine of a patient, and at least one container holding the liquid.
